(19) United States
(12) Patent Application Publication
(10) Pub. No.: US 2023/0181310 A1
(43) Pub. Date: Jun. 15, 2023

(54) CAPSULAR PROSTHESIS FOR POSTERIOR CHAMBER INTRAOCULAR LENS (IOL) FIXATION

(71) Applicant: Mark Michael Femandez, Norfolk, VA (US)

(72) Inventor: Mark Michael Femandez, Norfolk, VA (US)

(21) Appl. No.: 17/156,694

(22) Filed: Jan. 25, 2021

(51) Int. Cl. A61F 2/16

(52) U.S. Cl. CPC A61F 2/16; A61F 2002/16901; A61F 2002/16902; A61F 2220/0008; A61F 2230/0019; A61F 2002/0081

(57) ABSTRACT

An intraocular lens (IOL) prosthesis is implanted through one or more corneal incisions made within a predetermined surgical axis defining a plane that intersects the optical axis as well as the visual axis of the human eye. At least two looped sutures are fed through suture apertures and sutured to the sclera of the eye by which a center aperture of the prosthesis is suspended in the plane within the posterior chamber of the eye and centered on either the optical or visual axis. The prosthesis is made of a thin sheet of flexibly resilient material (e.g. silicone, polyimide, acrylic, polypropylene), and can be rectangular, triangular or of any suitable geometric shape. The center aperture has dimensions that facilitates optic capture or reverse optic capture of commercially available IOLs and includes vertex features for resisting movement of the haptics of the captured IOLs once captured therein.

### CROSS-REFERENCE TO RELATED APPLICATIONS

This application is related to the US Pat. App. titled “METHODS FOR SURGICAL IMPLANTATION OF A CAPSULAR PROSTHESIS FOR POSTERIOR CHAMBER INTRAOCULAR LENS (IOL) FIXATION,” and which is hereby incorporated herein in its entirety by this reference.

### FIELD OF THE INVENTION

The invention relates to intraocular lens (IOL) implantation, and more particularly to the fixation of IOL implants in situations where capsular support is inadequate, or non-existent.

### BACKGROUND OF THE INVENTION

Cataract surgery is one of the most frequently and successfully performed surgeries performed on the human eye. The American Society of Cataracts and Refractive Surgery (ASCRS) estimates that 3 million Americans undergo cataract surgery each year, with an overall success rate of 98 percent or higher. A cataract is simply defined by clouding or discoloration of the crystalline lens that makes it difficult to focus light onto the retina 30. When this occurs, a cataract surgeon removes the crystalline lens and replaces it with an artificial intraocular lens (i.e. IOL) that is able to properly focus light once again onto the retina (30, FIG. 1A) correctly.

FIG. 1A is a simplified illustration of the anatomy of the human eye 10. The crystalline lens 26 of the eye 10 has a nucleus 31 encased by a membranous bag-like structure called a capsule 24, which is divided into the posterior 28 and anterior 34 capsules. The capsule 24 lies within the anterior segment 19 of the eye 10, which is the front third of the eye 10 located in front of the vitreous humor 11, and includes the cornea 14, iris 12, ciliary body 21, and crystalline lens 26. The crystalline lens 26 is generally located posterior to the iris 12. The anterior chamber 16 is the space between the iris 12 and the cornea 14. The crystalline lens 26 is suspended in place within the posterior chamber 17 by fine suspensory fibers called zonules 22 originating from the ciliary body 21.

The crystalline lens 26 is generally aligned with the optical axis A-A′ 55. It extends through the geometric center of the cornea 16 to the geometric center of the retina 30, approximately halfway between the optic nerve 31 and the fovea 32. The optical axis 55 is defined by the geometric centers of cornea 16, pupil 20, and retina 30. However, the visual axis B-B′ 59 is the actual axis through which the human eye looks, which runs from a person's point of fixation to the fovea 32. The angle α between the optical 55 and visual 59 axes is about 5.2°.

A number of techniques are available to remove cataracts, and the one ultimately employed by the surgeon is dependent upon factors such as how advanced the cataracts are and the health of the patient's eyes generally. Phacoemulsification is the most commonly employed and desirable technique. The surgeon first tears a circular hole (i.e. capsulorhexis) (See 40, FIG. 1B) in the anterior capsule 34 to access the cataract. The crystalline lens 26 is loosened from the capsule 24 by injecting saline solution between the capsule 24 and the cataractous lens 26 material. The lens 26 material internal to the capsule 24 is liquified and aspirated from the eye using a phacoemulsification device (e.g. a metal cannula that vibrates at ultrasonic frequency). The device breaks up the cloudy cataract into tiny fragments that are removed from the eye 10 using suction.

As long as the capsule 24 remains largely intact other than the hole 40 (i.e. capsulorhexis) through which the affected crystalline lens 26 is removed, an IOL 70, 80 (such as the ones illustrated in FIGS. 2A, B) is inserted through the incision and capsulorhexis 40 and is implanted within the capsular bag 24 in place of the removed crystalline lens 26. Capsular placement or implantation is the optimal location anatomically for an IOL intended to replace the removed cataract. It provides optimal stability and permits the optic 72, 82 of the IOL to be located closest to the nodal point of the original nucleus 31 of the crystalline lens 26, through which the optical axis 55 of the eye passes.

There are many types of intraocular lenses 70, 80 currently available, and are typically either a single-piece design 70, or a three-piece design 80. The choice of lens is at least partially dictated by the therapeutic purpose to be served, as well as its suitability to the location within the eye where the IOL ultimately will be placed. IOL's all have an optic 72, 82 to focus the light on the retina 30 in lieu of the removed crystalline lens 26, and arms (or haptics) 74, 84 that provide a reactive force to help hold and center the optic 72, 82 in a fixed position, centered within either the optical axis 55 as illustrated in FIG. 1B, or the visual axis 59. Centration of the optic 72, 82 is important to obtaining desired visual acuity. Most lenses have been designed to be centered with the center of the pupil 20 (and thus the optical axis 55) even though the visual axis 59 does not pass through this point. For spheric and aspheric lenses, this does not affect the visual acuity significantly. For newer lens technologies such as multifocal lenses, it may be more desirable to center the optic with the visual axis 59 for optimal visual acuity.

Single piece IOL's 74, FIG. 2A are usually made entirely (both optic 72 and haptics 74) from hydrophobic or hydrophilic acrylic. As a result, single piece IOLs have haptics 74a, b that are soft and broad. They are often preferred for placement in reasonably intact capsules 34. Single-piece tonic lenses are designed to correct for a patient's astigmatism. The optics 82 of three-piece IOLs 80, FIG. 2B are made from acrylic, silicone, or another suitable elastomer, and have haptics 84a, b that are made separately from the optic 82 and attached thereto. The haptics 84a, b are typically made of a different material such as polymethyl-methacrylate (PMMA) or polypropylene. Both are suitable for placement of an optic within the capsule as illustrated in FIG. 1B.

For many reasons, the capsule 24 is not always left sufficiently intact to support implantation of the IOL 70, 20 within the capsule 24 as shown in FIG. 1B. For example, a second surgical technique called extracapsular cataract surgery is sometimes employed in situations where the condition of the eye prevents the use of the more desirable phacoemulsification. One such situation is when the cataracts are more advanced, which renders them too dense for phacoemulsification. Extracapsular surgery requires a larger incision in the anterior capsule 34, which requires sutures for proper healing of that larger incision. In addition, it is not uncommon that during cataract surgery or long after, a number of complications can occur that can make it impossible to securely place an artificial IOL lens 70, 80 within the capsule. For example, the posterior capsule 28 can rupture during surgery such that a large hole (in addition to the surgically created capsulorhexis 40) in the capsular bag 24 precludes placing an IOL within it.

In cases where capsular placement of an IOL is not possible, a three-piece IOL 70 can be placed within the ciliary sulcus 18. FIG. 1C illustrates such a placement. The haptics 84a, b can be seen located in the sulcus 18, and the optic 82 is located anterior to the anterior capsule 34 and the capsulorhexis 40 made therein to remove the cataract. Unfortunately, for this type of placement, the long term centration of the optic 82 of the IOL 80 to the optical axis 55 can become compromised. Moreover, the haptics 84a, b can migrate and rub against the iris 12, causing irritation thereto and depigmentation thereof. Patients often require a second procedure months or years after the first surgery to suture the lens 80 to the iris 12 or sclera 36 so that it does not fall into the posterior chamber (not shown), and to recenter the lens 80 to the optical axis 55 so that it properly focuses light onto the retina 30.

If the anterior capsule 34 is reasonably intact, and the zonules 22 are able to still support the anterior capsule, an alternative technique for ciliary sulcus 18 placement (not pictured) can be used called reverse optic capture. In this technique, a three piece IOL (80, FIG. 2B) can be placed such that the haptics 84a, b are anterior to the anterior capsule 34, FIG. 1C and the optic 82 of the IOL 80 is then prolapsed posteriorly so that the optic 82 is forced through an intact capsulorhexis 40 in the anterior capsule 34 and is held in place thereby. Placement within the ciliary sulcus 18 via reverse optic capture is a more stable technique by which to achieve an IOL 80 with proper centration with respect to the optical axis 55 (as defined by the iris 12 and the pupillary border 44) notwithstanding a damaged posterior capsule 24, than is the simpler sulcus placement of FIG. 1C.

Another technique used for anterior segment 19 placement of an IOL 90 is to suture a three piece IOL to the iris 12. Although a relatively good technique, it is technically difficult with a lengthy procedure that includes a steep learning curve. In addition to being difficult to perform, it is not unusual for the lens to chafe the iris 12, causing inflammation or for the lens to dislocate.

In some situations, the entire capsule 24 complex (anterior 34 and posterior 28 capsule) is damaged and/or removed (see FIG. 1D), or the zonules 22 are damaged so extensively that ciliary sulcus 18 placement of the IOL 80, with or without reverse optic capture simply cannot be performed. Thus, the next available mode of IOL (90, FIG. 1D) placement will typically be within the anterior chamber 16 of the eye 10. Those of skill in the art will appreciate that FIG. 1D is intended to illustrate anterior IOL 90 placement in general, and not the fine details of any specific such anterior chamber lens design or technique. Currently, the most common way to address this complication is to make an even larger incision by which to place an anterior chamber lens (ACIOL) anterior to the iris 12. While this technique is relatively simple, the large incision slows healing and the technique is more likely to cause failure of the cornea 14, requiring corneal transplantation later in life.

In another known technique for anterior chamber 16 placement, an IOL 90 can be sutured directly to the white part of the eye (i.e. sclera 36). While this technique of anterior chamber placement does not damage the cornea 14, it is often performed using a larger rigid lens which requires a commensurately larger incision. Because almost all lenses used for this technique have only two haptics, many of which are designed with varying angulation, only two effective points of contact exist between the IOL and the sclera 36, making it easy for the surgeon to inadvertently place the lens 90 in a way that it will rotate and rub against the iris 12. This can lead to iris chafe and inflammation within the eye. Finally, because many of the techniques discussed above require suturing the lens to the eye, it renders any efforts to replace those lenses a significant surgery in and of itself.

It would be desirable to avoid IOL placement after cataract surgery anterior to the capsule 24 in situations where the capsule 24 is not able to support placement therein, and particularly to avoid placements within the anterior chamber 16. Placement within the capsule 24 is the natural position for lens placement and avoids the complications that can occur for placements within the anterior chamber 16, and also within the sulcus 18. It would also be desirable to minimize the invasiveness of procedures required to replace previously implanted lenses. It would be further desirable to facilitate a more uniform technique for lens replacement regardless of the type of IOL used, and to provide more freedom to achieve a desired centration of the optic.

### SUMMARY OF THE INVENTION

A capsular prosthesis of the invention is disclosed that is configured to be implanted to support placement of IOLs in a position that substantially corresponds to the location of the naturally occurring crystalline lens provided by an intact capsule of the human eye prior to its removal. The capsular prosthesis can be implanted to essentially replace the capsule in situations where the patient's natural capsule has been rendered incapable of providing the structural support necessary to maintain proper centration of an IOL implanted therein. A method of implanting the prosthesis is further disclosed.

In one aspect of the invention, embodiments of a capsular prosthesis are disclosed for capturing and supporting an intraocular lens (IOL) within the posterior chamber of an eye. The intraocular lens has an optic and at least two haptics. The prosthesis includes a sheet of substantially bioinert material The sheet has two substantially planar surfaces separated by the thickness of the material. The sheet also includes a center aperture and a plurality of suture apertures configured to receive at least two loops of suture. Each loop of suture has two ends. When the two ends of each of the at least two loops of suture are passed through the sclera of an eye approximately 180 degrees from one another along a predetermined surgical axis C-C′ 60, the two loops of suture permit adjustment of the sheet along the surgical axis C-C′ 60 to achieve centration of the center aperture to a predetermined axis of the eye. The centration becomes fixed when each of the two ends of the loops are then fixedly attached to the sclera.

In an embodiment, the center aperture is configured to capture the optic of an IOL against a portion of a first one of the two planar surfaces, and permits the haptics of the IOL to be passed through the center aperture to apply reactive force to a second one of the planar surfaces to retain the optic against the first surface and centered to the predetermined axis.

In a further embodiment the center aperture defines at least two vertex features, each of the pair of vertex features being defined by the aperture and located proximally to one of the opposite ends of the sheet. When supporting an IOL thereon, each of the vertex features is configured to capture one of the haptics of the IOL as it passes through the center aperture to resist further movement of the haptic.

In an embodiment, the sheet is substantially rectangular in geometry.

In a further embodiment, the at least three suture apertures include at least two pairs of suture apertures. One pair located at a proximal end of the sheet is configured to receive the two ends of a proximal one of the at least two loops of suture. A distal pair is located at a distal end of the sheet and is configured to receive the two ends of a distal one of the two loops of suture.

In a still further embodiment, each of the at least two pairs of suture apertures are located proximally to a different one of the four corners of the sheet.

In a further embodiment, each of the four corners of the sheet is rounded.

In another embodiment, the sheet is substantially triangular in geometry.

In a further embodiment, the at least three suture apertures include at least one suture aperture located proximally to a different one of each vertex of the sheet, each suture aperture configured to receive at least one of the two ends of one of the at least two loops of suture.

In further embodiment, each vertex of the sheet is rounded.

In yet another embodiment, the bioinert material comprising the sheet is sufficiently deformable to permit folding of the sheet under a folding force during insertion into the eye through an incision, but sufficiently resilient such that it substantially unfolds back to its full geometry after the force is removed.

In a still further embodiment, the bioinert material comprising the sheet is one of: silicone, polyimide, acrylic.

In a further embodiment, the IOL is configured to be supported by the sheet is a three-piece IOL and the sheet is configured to support the three-piece IOL through reverse optic capture, wherein the optic is retained against a posterior one of the two planar surfaces of the sheet, and the haptics are passed through the center aperture to make contact with an anterior one of the two planar surfaces.

In an embodiment, the sheet has a length of about 11 mm, a width of about 7 mm and a thickness of about 0.25 mm.

In an embodiment, the center aperture has an inner length of about 8 mm between the vertex features and an internal width of about 5 mm.

In an embodiment, the predetermined axis of the eye is the optical axis.

In another embodiment, the predetermined axis of the eye is the visual axis.

In another embodiment, the dimensions of the sheet and the center aperture are configured to enable substantial concentric alignment of the supported IOL captured thereon with the optical axis of the eye when the first and second sutures are adjusted.

In a still further embodiment wherein the sheet and the center aperture are configured to enable substantial concentric alignment of the supported IOL captured thereon with the optical axis of the eye, by adjusting the first and second looped sutures before the surgical fixation of their respective ends in the sclera.

In another embodiment, the IOL supported by the sheet is a one-piece IOL, and the sheet is configured to support the one-piece IOL through optic capture, wherein the optic is retained against an anterior one of the two planar surfaces of the sheet, and the haptics are passed through the center aperture to make contact with a posterior one of the two planar surfaces.

In yet another embodiment, the IOL is a toric lens and the predetermined surgical axis is substantially equal to an axis of astigmatism of the eye.

### BRIEF DESCRIPTION OF THE DRAWINGS

FIG. 1A is a simplified cross-sectional side view illustration of the anatomy of the human eye;

FIG. 1B is a simplified cross-sectional side view illustration of a human eye from which a cataract has been surgically removed and replaced with an intraocular lens (IOL) that has been implanted in the capsule using techniques known to those of skill in the art;

FIG. 1C is an image of a human eye from which a cataract has been surgically removed and replaced with an intraocular lens (IOL) placed in the sulcus in accordance with techniques known to those of skill in the art;

FIG. 1D is a simplified cross-sectional side view illustration of a human eye from which a cataract has been surgically removed and replaced with an intraocular lens (IOL) that has been surgically implanted into the anterior chamber in accordance with techniques known to those of skill in the art;

FIG. 2A is a plan view of a single piece IOL known to those of skill in the art;

FIG. 2B is a plan and side view of a three-piece IOL known to those of skill in the art;

FIG. 3A is a plan view of the embodiment of the capsular prosthesis of the invention;

FIG. 3B is a side view of the embodiment of the capsular prosthesis of the invention;

FIG. 4A is an elevated anterior view of the embodiment of FIGS. 3A, B of the invention with a three-piece IOL reverse optically captured thereon;

FIG. 4B is a side view of the embodiment of FIGS. 3A, B of the invention with a three-piece IOL reverse optically captured thereon;

FIG. 5 is a plan view of an embodiment of the capsular prosthesis of the invention;

FIG. 6A is a is an elevated anterior view of the embodiment of FIG. 5 with a one-piece IOL optically captured thereon;

FIG. 6B is a is an elevated posterior view of the embodiment of FIG. 5 with a one-piece IOL optically captured thereon;

FIG. 7 is a plan view of a rectangular embodiment of the capsular prosthetic, having been surgically implanted within the eye, supporting a one piece IOL through optic capture;

FIG. 8 is a plan view of a triangular embodiment of the capsular prosthetic, having been surgically implanted within the eye, supporting a three piece IOL through reverse optic capture;

FIG. 9 is a plan view of a rectangular embodiment of the capsular prosthetic, having been surgically implanted within the eye using an alternate procedure that uses only one corneal incision;

FIG. 10A is a diagnostically produced visual representation of a patient's axis of astigmatism; and

FIG. 10B is a plan view of an adjusted surgical placement of the embodiment of the prosthesis of the invention of FIGS. 3A, B and 4A, B having a one-piece toric IOL optically captured thereon, to compensate for the angle of astigmatism of FIG. 10A;

FIG. 11 is a side view of a human eye within which the embodiment of the prosthesis of the invention of FIGS. 3A, B and 4A, B has been surgically implanted to support a three-piece IOL having been secured thereon using reverse optic capture.

### DETAILED DESCRIPTION

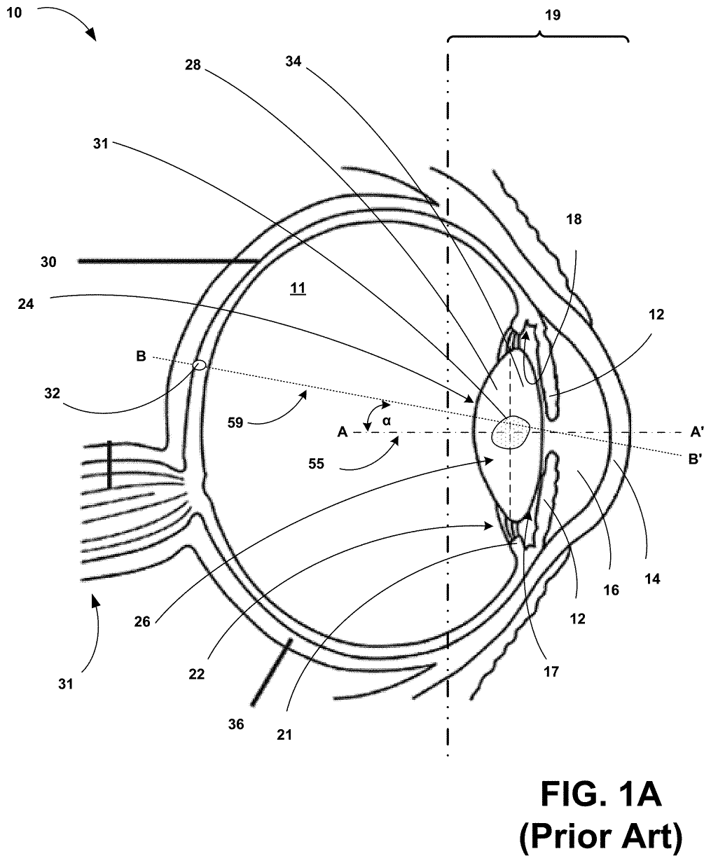

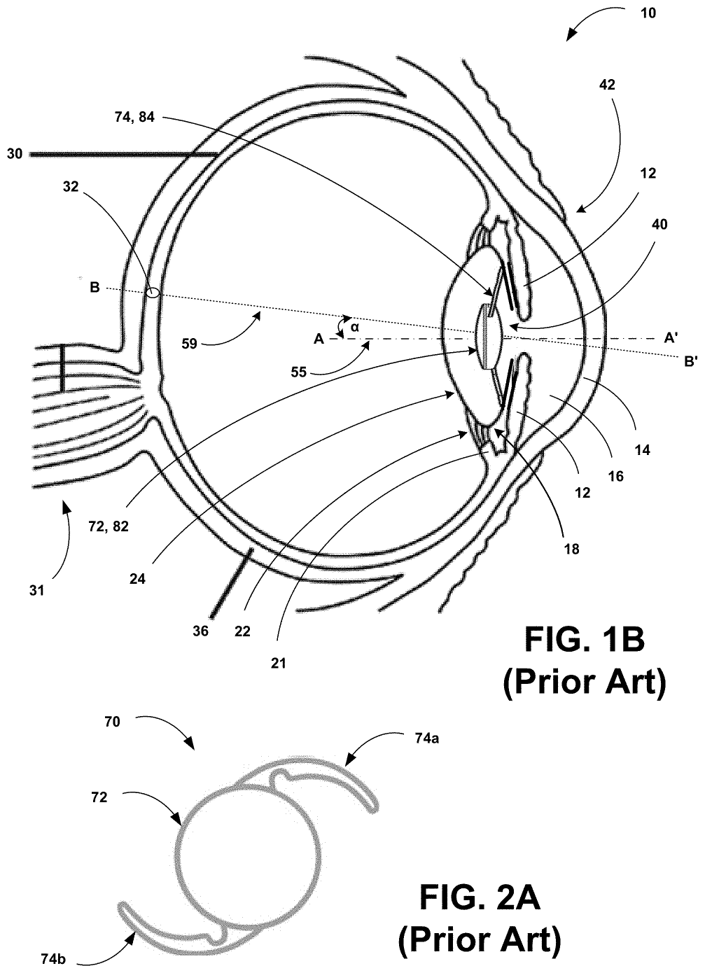

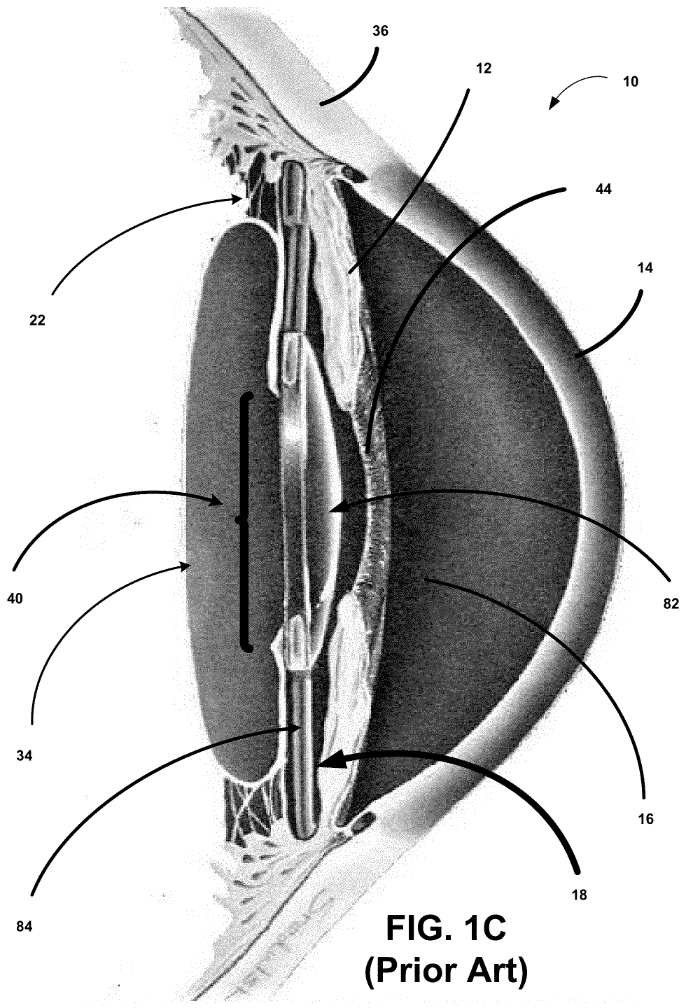

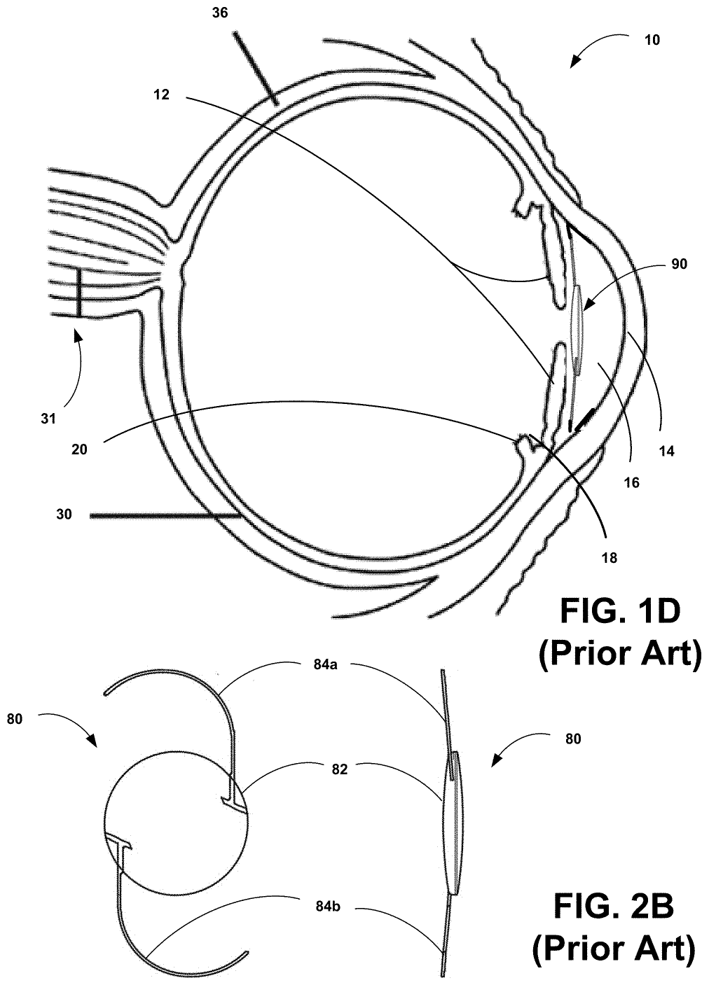

Embodiments 100, 200 of methods for surgically implanting a capsular prosthesis are disclosed that are configured to receive and support commercially available single and three-piece IOL's 70, 80 (FIGS. 2A, 2B) via their haptics 74, 84 (FIGS. 2A, 2B) by way of a prosthetic optic capture to secure the lens 70, 80 (FIGS. 2A, 2B) to the prosthesis and to hold accurate centration of its optic 72, 82 (FIGS. 2A, 2B) with desired axis of the eye thereby. This eliminates the need to suture the haptics 74, 84 (FIGS. 2A, 2B) themselves to either the sclera 36, FIG. 1A or the iris 12, FIG. 1A as is often required of anterior chamber 16, FIG. 1D and some sulcus 18, FIG. 1C placement techniques in lieu of capsular implantation (FIG. 1D) when it is not practicable. Moreover, the methods of implanting the prosthesis 100, 200 of the invention serve to normalize placement of the various lens designs with their haptics of varying materials, lengths, and degrees of angulation. The methods of surgical implantation of prosthesis 100, 200 simplify placement of the lens optic 72, 82 planar to the iris 12 and with substantially perfect centration to a desired axis of the eye 50, such as optical axis 55 or visual axis 59 regardless of the design of the IOL used.

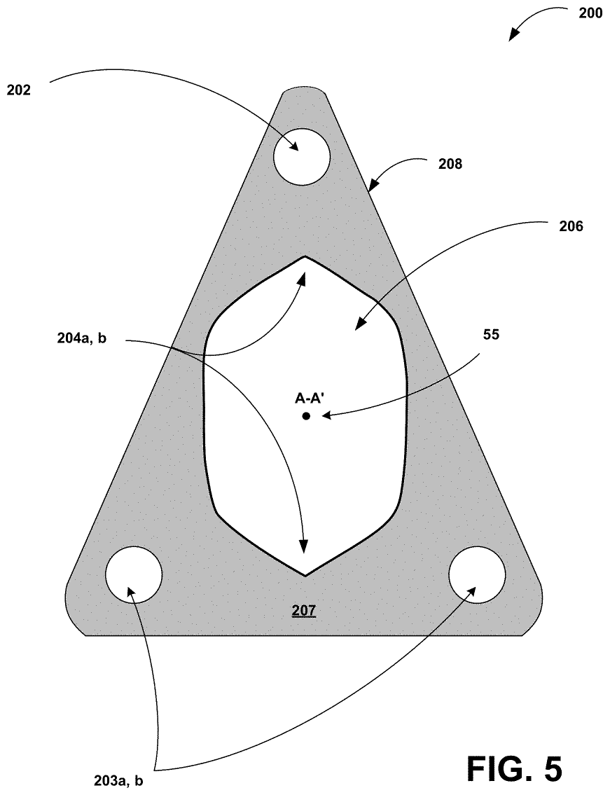

The prosthesis 100, 200 (and associated methods of its implantation) provide a plurality of points of contact greater in number than just the two typically provided by the haptics of an IOL alone, rendering the IOL largely immune from torqueing after implantation, as well as eliminating the need for post-operative adjustments of the IOL to achieve optimal centration with the eye's optical 55 or visual 59 axis. These points of contact are made by way of at least two sutures, one proximal and one distal to the surgeon, which are looped through prosthesis 100, 200 and introduced through the sclera 36. These points are predetermined by the surgeon to achieve a desired surgical axis C-C′ (60, FIGS. 5A, 6A, 8) for placement of the prosthesis that defines a plane that is perpendicular to the desired axis to which the IOL 70, 80 is to be centered. Centration can be achieved by pulling on the two sutures before they are surgically fixed (e.g. tied to create knots, subjected to heat cautery, etc.) within the sclera 36.

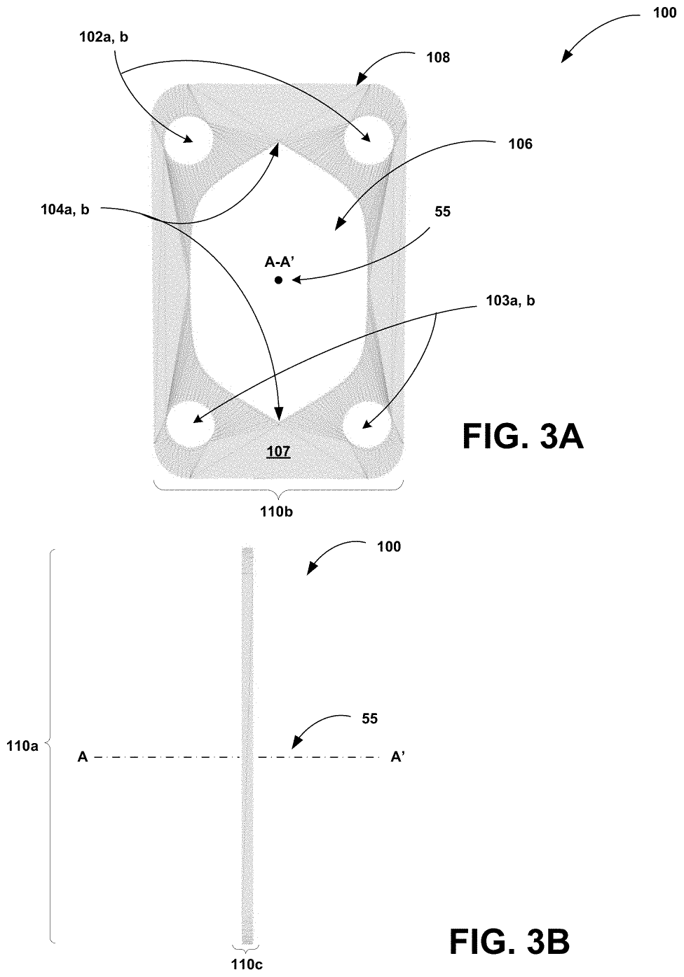

The capsular prosthesis 100, 200 is surgically secured within the posterior chamber 17 (in the space normally occupied by the anterior capsule 34) through embodiments of the surgical implantation methods of the invention. As a result, the prosthesis of the invention 100, 200 of FIGS. 3A, 3B, 4A, 4B, 5, and 6A, B has been configured to support a three-piece IOL having been secured thereon using reverse optic capture, or optic capture. This enables standard IOLs to be placed in substantially the same concentric alignment as that previously provided by the patient's pre-operative capsule for the removed cataract. Surgical implantation of the capsular prosthesis thereby eliminates the need for virtually all of the less than ideal placement techniques of IOLs in the ciliary sulcus 18 or the anterior chamber 16, and particularly in situations where the capsule 24 is not sufficiently sound to support capsular implant of an IOL 70, 80 within the naturally occurring capsule 24.

The prosthesis 100, 200 of the invention can essentially replicate sulcus 18 placement of three-piece IOLs 80 with reverse optic capture, in that the center aperture 106, 206 of prosthesis 100, 200 of the invention acts in lieu of an intact capsulorhexis 40 of an anterior capsule when using reverse optic capture for a sulcus placement of an IOL. It can also be used to accomplish optic capture of one-piece IOLs 80 by capturing the optic 82 on the anterior side of the prosthesis and prolapsing the haptics to the posterior side of the prosthesis. The haptics 84 are placed though the center aperture 106 and forward of the anterior capsule 34, and the optic 82 of the three-piece lens 80 is captured against the prosthesis similar to the manner in which it is captured if it were prolapsed through the capsulorhexis of anterior capsule 34. Alternatively, if a one-piece IOL 70 is used that cannot safely be placed in a reverse optic capture orientation, the haptics 74 can be prolapsed posterior to the prosthesis 100, 200 with the optic 72 being placed anterior to the prosthesis 100, 200.

Existing methods of lens placement and fixation, particularly within the anterior chamber 16, involve securing the IOL to structures in the eye 50 itself using sutures. Thus, when replacing that IOL when indicated by, for example, a poor refractive outcome, such replacement becomes a major surgical procedure to remove the sutures of the IOL to be replaced, and then suturing in a new one. The prosthesis 100, 200 of the invention facilitates easy lens replacement through a single small incision, because the implanted prosthesis 100, 200 itself does not have to be removed to replace the IOL. Replacement simply requires that the existing IOL supported by the prosthetic 100, 200 be removed and replaced with a new IOL being supported by the previously implanted prosthetic. Thus, easy fixation of various commercially available IOL designs to the prosthesis 100, 200 of the invention renders IOL removal and replacement simple and easy.

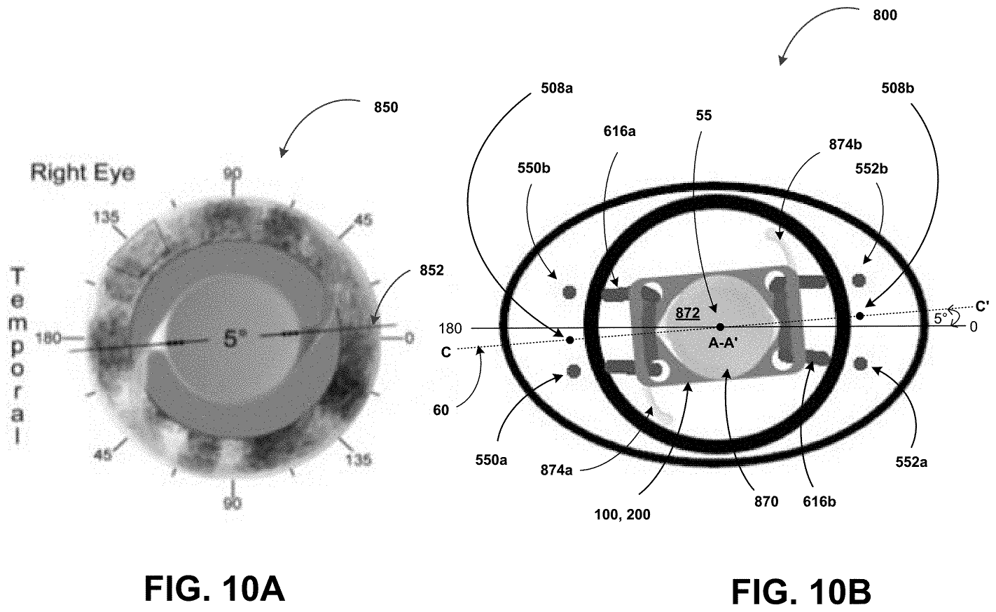

Easy removal also facilitates the use of advanced technology IOLs, like multifocal and trifocal lenses. While these lenses provide a greater range of focus, they are also less forgiving of decentration or retinal issues. Likewise, the ability to rotate the surgical axis 60 in performing the methods of surgical implantation of the invention also permits easier centration of the IOLs with the visual axis 59. For example, a multifocal IOL fixated within the prosthesis of the present invention, rather than directly to the iris 12 or sclera 36, can be easily replaced with a mono-focal IOL without extensive damage to the supporting structures of the eye 10. Those of skill in the art will appreciate that the methods of surgical implantation of the prosthesis 100, 200 of the invention is not limited to lens replacement necessitated by the surgical removal of cataracts. As is illustrated in FIGS. 10A and B, a desired surgical axis 1510 can be established that matches the axis of astigmatism 900 of an eye 800 to facilitate easier placement of single-piece toric lenses 872 as well.

FIGS. 3A, B illustrate an embodiment 100 of the capsular prosthesis of the invention. In an embodiment, the prosthesis 100 is a thin rectangular sheet 108 of bioinert, flexible (yet resilient) material having two planar faces 107 that are substantially identical. The sheet has two sets 102a, b and 103a, b of suture apertures through the sheet 108 at its corners. The sheet 108 has an aperture 106 substantially centered within that is large enough to provide an optical line of sight along optical axis A-A′ 55 for the optics of most commercially available intraocular lenses (IOLs). Center aperture 106 further includes vertex features 104a, b suitable for capturing haptics of the IOLs passed therethrough to resist them from sliding once captured therein.

In an embodiment, the sheet 108 can have a length 110a of approximately 11 mm, a width 110b of approximately 7 mm, and a thickness 110c that can be approximately 0.25 mm. In an embodiment, center aperture 106 can have an internal length of about 8 mm between vertex features 104, and an internal width of about 5 mm. The diameter of the suture apertures 102a, b and 103a, b can be about 1.5 mm. Those of skill in the art will recognized that these dimensions may be varied to fit a range of commercially available lenses, sutures, and needles. The thickness 110c of the sheet 108 will vary depending upon the material from which the sheet is made. The sheet can be made of bioinert materials including but not limited to, silicone, polyimide, acrylic or the like. The sheet 108 should be flexible enough that it is foldable, so that it can be made small enough to be inserted into the eye through a primary clear corneal incision of about 2-3 mm. It should also be sufficiently resilient to re-establish its full original dimensions for proper deployment once inserted into the eye. Those of skill in the art will appreciate that the height of the sheet 108 will be dictated by the anatomy of the eye, such that the sheet 108 should be operable to capture and support the optic of the lens, approximately centered on the center aperture 106, and properly aligned with the visual axis 55. The sheet 108 does not have to be particularly rigid because it is being sutured at its four corners, which allows it to be suspended like a trampoline and stretched to sufficiently supportive rigidity within the appropriate plane.

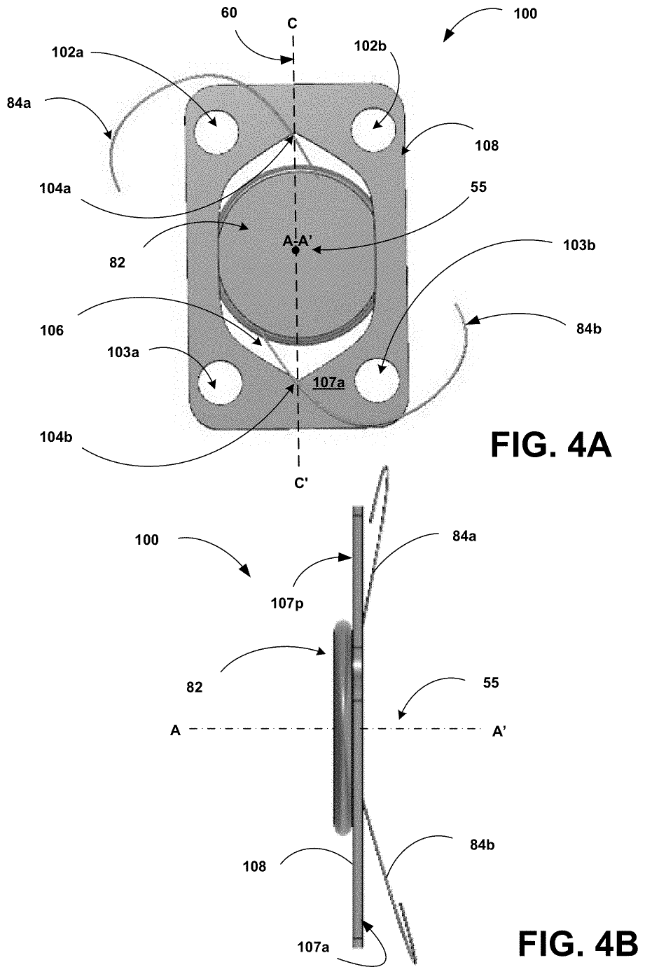

FIGS. 4A, B illustrate a view of what is defined as the anterior surface 107a of the prosthesis 100 from the perspective of a surgeon. The three-piece IOL 80, FIG. 2B mounted on the prosthesis 100 having optic 82 and haptics 84a, b. The IOL 80 is mounted in a reverse optic capture configuration, with its haptics 84a, b placed forwardly through aperture 106 from the posterior side and captured within vertex features 104a, b respectively on the anterior surface 107a. Optic 82 is substantially centered behind aperture 106 on what is the posterior surface 107p from the perspective of a surgeon and has optical line of sight along axis A-A′ 55. FIG. 4A shows a surgical axis C-C′ 60 aligned with the optical axis 55.

FIG. 4B shows a side view of the reverse captured IOL 80, whereby the optic 82 has been prolapsed through the aperture 106 such that haptics 84a, b exert a force on the anterior surface 107a that pulls the optic 82 against the posterior surface 107p of the sheet 108. This works much the same way as does a sulcus placement of such a lens using reverse optic capture, wherein the optic 82 is prolapsed into the capsulorhexis 40 into the anterior capsule 34, the haptics 84a, b disposed in the sulcus 18 and pulling the optic 82 against the inside surface of the anterior capsule 34 defining the capsulorhexis 40.

FIG. 5 illustrates an alternate embodiment 200 of the prosthesis of the invention that is triangular in geometry rather than rectangular. This triangular embodiment 200 has three, rather than four, suture apertures 202, 203a and 203b each located proximally to one of the three vertices of the triangular sheet 208. The three vertices 202, 203a and 203b are rounded off to avoid creating sharp points that could irritate or potentially damage structures in the eye during implantation. The thin rectangular sheet 208 is also made of a bioinert, flexible (yet resilient) material having two planar faces 207 that are substantially identical. The triangular sheet 208 has an aperture 206 substantially centered within that is large enough to provide an optical line of sight along a desired axis of the eye 50, (e.g. optical axis A-A′ 55 or visual axis B-B′ 59 for the optics of most commercially available intraocular lenses (IOLs). Center aperture 206 further includes vertex features 204a, b suitable for capturing haptics of the IOLs passed therethrough to resist them from sliding once captured therein. In an embodiment, the dimensions of the triangular sheet 208 can be scaled as necessary to accommodate the inside dimensions of the center aperture 206.

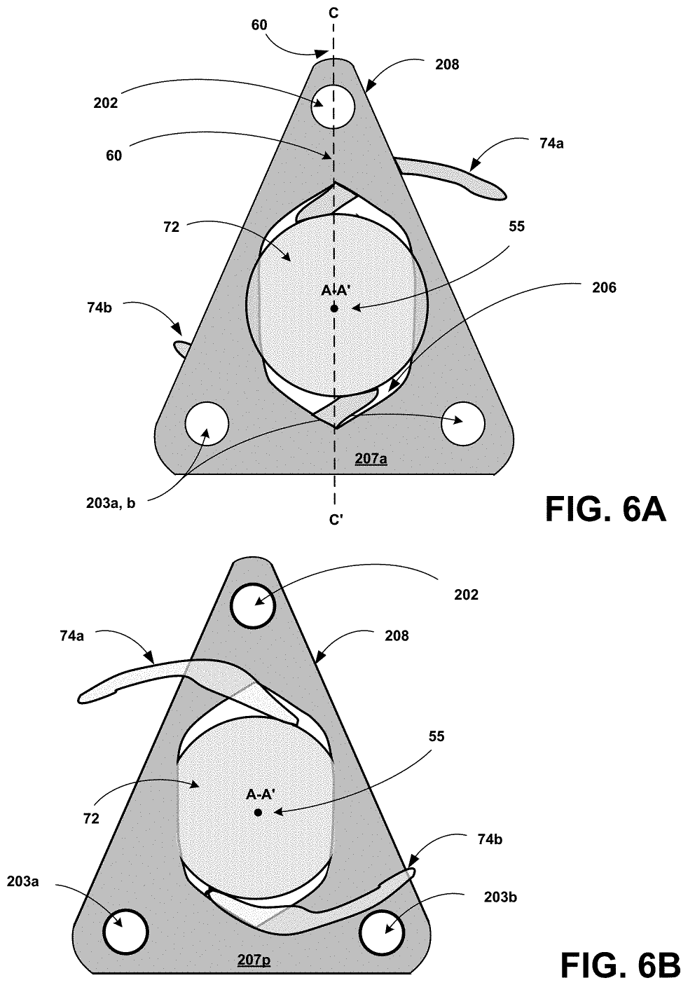

FIG. 6A illustrates a view of what is defined as the anterior surface 207a of the prosthesis 200 from the perspective of a surgeon. A one-piece IOL 70, FIG. 2A is mounted on the prosthesis 200 having optic 72 and haptics 74a, b. The IOL 70 is shown mounted on prosthesis 200 using an optic capture, where its haptics 72a, b are placed rearwardly through aperture 206 and emerging from the aperture 206 and captured within vertex features 204a, b respectively on the posterior side and surface 207p. Optic 72 is substantially centered on aperture 206 on what is the anterior surface 107a from the perspective of a surgeon and has optical line of sight along axis A-A′ 55 of an eye 50. Those of skill in the art will appreciate that centration of the optic 72 can be made on any desired axis, including the visual axis B-B′ 59. FIG. 6B shows a posterior view of the captured IOL 70, whereby the haptics 74a, b have been prolapsed through the aperture 206 from the anterior side such that haptics 74a, b exert a force on the posterior side surface 207p that pulls the optic 72 against the anterior surface 107a of the sheet 208 and maintains its position.

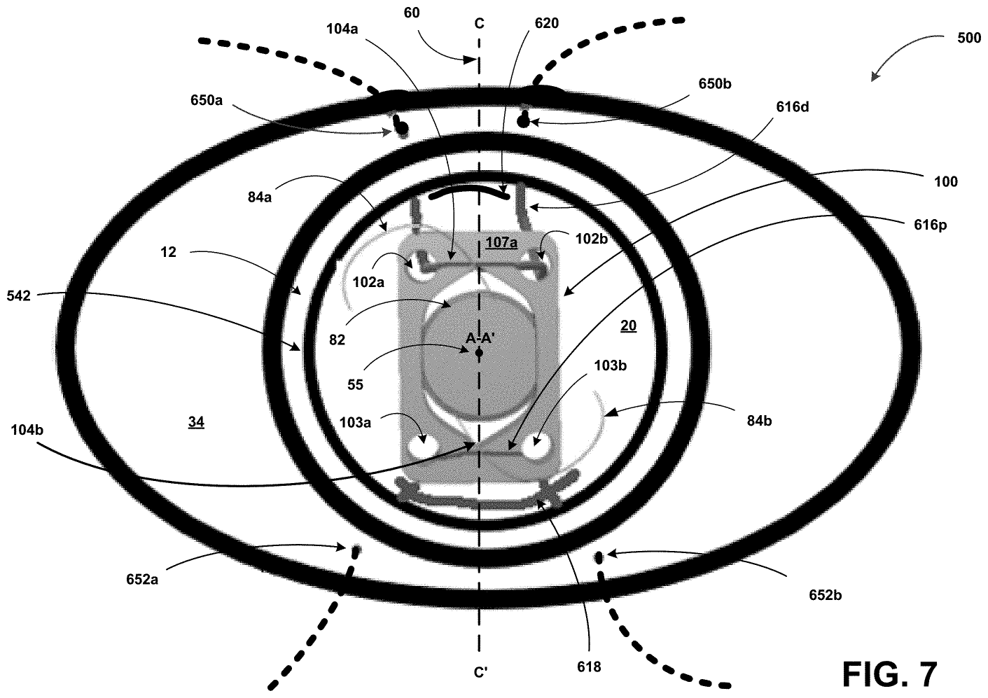

FIG. 7 illustrates prosthesis 100 and a reverse optically captured three-piece IOL having optic 82 and haptics 84a, b mounted thereon, having been implanted in the posterior chamber 18 of eye 500 within the space that was once the approximate location of the anterior capsule 34 in accordance with an embodiment of surgical methods of the invention. Two transscleral sutures 616p (i.e. posterior) and 616d (i.e. distal) are shown, which are passed through sclera 36 at sclerotomies 652a, b and 650a, b respectively, and looped through the suture apertures 103a, b and 102a, b of prosthesis 100 respectively. Each pair of ends of the looped sutures 616p, d respectively are ultimately surgically secured to the sclera 36 at the pairs of sclerotomy points 652a, b and 650a, b at the bottom and top of the eye 500 respectively, each pair being approximately 180 degrees from one another along desired surgical axis C-C′ 60.

As illustrated in FIG. 7, the prosthesis 100 has been inserted through one or more corneal incisions 618, 620 made within a predetermined surgical axis C-C′ 60 at which the IOL 80 is to be placed. Predetermined surgical axis C-C′ 60 defines a plane that intersects optical axis A-A′ 55 as well as the visual axis B-B′ 59. The limbus 542 of the eye 500 forms the border between the transparent cornea and opaque sclera 34, contains the pathways of aqueous humor outflow, and is the site of surgical incisions for cataract and glaucoma (hence being referred to as the surgical limbus). Both ends of each pair (the dotted portions of the looped sutures can be trimmed after being surgically affixed to the sclera 36 of the distal 616d and proximal 616p looped sutures can be pulled to adjust the position of the prosthesis 100 along the surgical axis C-C′ 60 as needed to substantially center the center aperture 106 of the prosthesis 100 to the desired (optical 55 or visual 59) axis of the eye 500. Once centration of the center aperture is achieved with the desired axis, each pair of ends of the transscleral looped sutures 616p, d can either be tied, or subjected to heat cautery to make thickened flanges to secure the sutures in place within the sclera 34.

With the prosthesis 100 now securely centered within the eye 500, a three piece intraocular lens 80 can be inserted into the eye through a primary incision 618 using a standard lens insertion cartridge (not shown) known to those of skill in the art. A Sinskey hook or other second instrument can be used as known in the art to manipulate the optic 82 so that its longitudinal edges are posterior to the prosthesis 100 and in contact with a posterior facing surface of the sheet 107p of the prosthesis 100, leaving the haptics 84a, b anterior to the prosthesis 100 and captured within the vertex features 104 to make contact with, and apply a retention force to, the anterior surface 107a of the prosthesis.

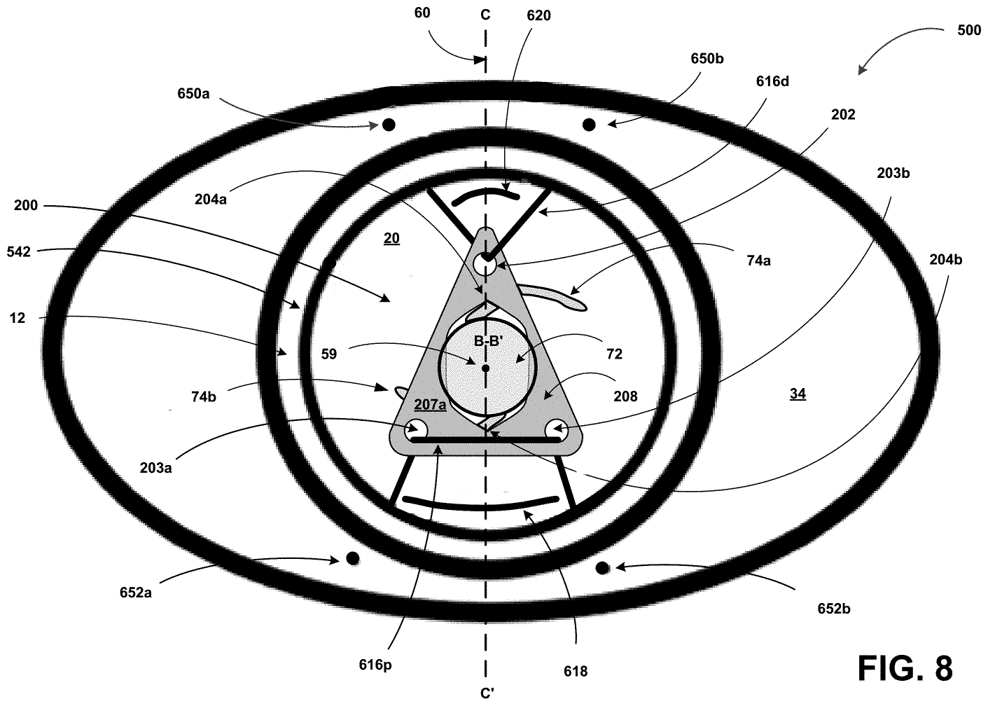

FIG. 8 shows triangular embodiment 200 of the prosthesis of the invention with an optically captured one-piece IOL having optic 72 and haptics 74a, b mounted thereon, having been implanted in the posterior chamber 18 of eye 500. The primary difference from the rectangular embodiment 100 is that the distal transscleral loop suture 616d is passed through a single suture aperture 202 at the vertex of triangular sheet 208. Each pair of ends of the looped sutures 616p, d respectively are ultimately surgically secured to the sclera 36 at the paired sclerotomy points 652a, b and 650a, b at the bottom and top the eye 500 respectively, after centering the center aperture 206 on the visual axis in this case. Each pair of ends being approximately 180 degrees from the other along desired surgical axis C-C′ 60.

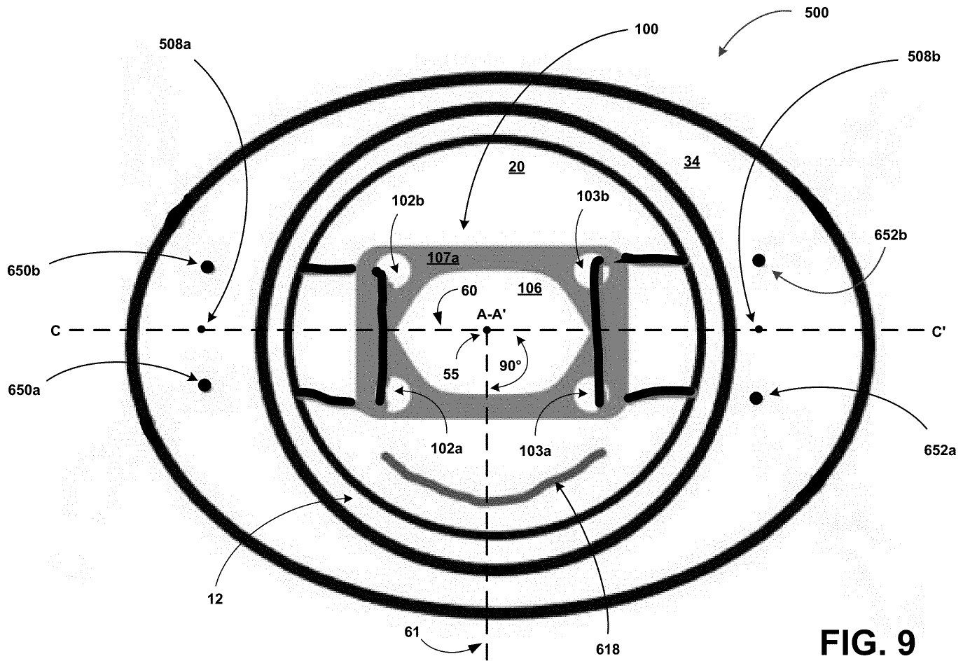

FIG. 9 illustrates prosthesis 100, 200 having been implanted using a single corneal incision 618, thereby eliminating the need for the secondary corneal incision 620 of FIGS. 7 and 8. This technique can also eliminate the need to cannulate the second transscleral suture (616p, FIGS. 7 and 8) within the eye 500. In this embodiment, both transscleral double armed sutures 616a, b can be looped through the suture apertures of prosthesis 100, 200 outside of the eye. The predetermined surgical axis C-C′ 60 has been rotated counterclockwise 90 degrees and is still perpendicular to the optical axis A-A′ 55. This is necessary because cannulating sclerotomies at the bottom of the surgical axis 60 through incision 618 would be exceedingly difficult. In this surgical method, the primary incision 618 is therefore bisected by an axis of incision 61 that is substantially perpendicular (i.e. at 90°) to the predetermined surgical axis C-C′ 60. Because incision 618 can be made over 360 degrees of the cornea, the surgical axis C-C′ 60 can be at any rotational position, which can be very useful for placing non-spherical lenses.

Thus, embodiments of the surgical method of the invention permit the prosthesis 100, 200 of the invention to be surgically implanted at any predetermined angle of orientation of the surgical axis C-C′ 60 over the 360° around virtually any axis, but particularly the optical axis A-A′ 55 or the visual axis B-B′ 59. This makes implantation of non-spherical lenses, such as a toric lens 870 that is designed to correct a person's astigmatism easier to implement. FIG. 10A presents a visual representation of a patient's astigmatism commonly produced by a diagnostic instrument. The astigmatism is presented as an angled axis of astigmatism 852 centered on the optical axis of the eye 800. FIG. 10B represents implantation of the prosthesis 100, 200 using a surgical axis C-C′ 60, predetermined to be substantially the same as the axis of astigmatism 852.

By orienting the prosthesis 100, 200 in accordance with the axis of astigmatism 852, the surgeon does not have to provide a correct orientation of the non-spherical lens. The surgeon must only orient the toric lens optic 872 with the center aperture 106 of the prosthesis, in accordance with standard orientation established by the manufacturer for optic capture within the prosthesis 100, 200. The standard orientation of the IOL 870 can be normalized to that disclosed in FIGS. 6A, 6B and 8, with the haptics 874a, b aligned to be captured by vertex features 104, 204 of prosthesis 100, 200. Because toric lenses 870 are typically single piece lenses, they will be captured using optic capture as is also illustrated in FIGS. 6A, 6B and 8.

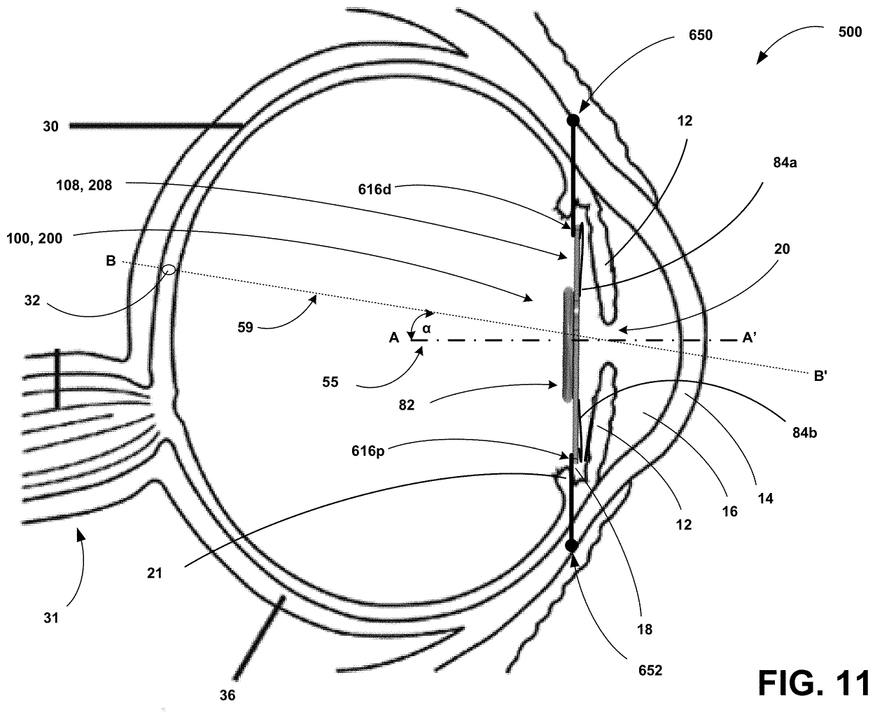

FIG. 11 illustrates a side view of the implanted prosthesis 100. It can be seen that the precise location of the prosthesis mounted lens along the optical axis A-A′ 55 (or the visual axis B-B′ 59) is not critical but should be sufficiently posterior to the iris 12 so that the haptics 84a, b can avoid contact with the iris 12 when reverse optically captured. The optic 82 should be positioned to be substantially planar with the iris and substantially concentrically aligned with the desired axis (e.g. A-A′ 55 or visual axis B-B′ 59) such that the suture 616p, d positions defined by the proximate 352a, b and distal 350a, b sclerotomy points are located anterior to the ciliary bodies 20. This of course defines the relative position of the optic 80 along the optical axis A-A′ 55. Those of skill in the art will recognize that other locations for the sclerotomy points (e.g. posterior to the ciliary bodies 21) resulting different positions of the optic 82 along the optical axis A-A′ 55, or to rotate the prosthesis and therefore the optic 82 may be also desirable, such as rotating the desired surgical axis C-C′ 60 to center the optic 82 on the visual axis B-B′ 59.

Those of skill in the art will recognize that certain modifications of the embodiments disclosed herein can be made without exceeding the intended scope of the invention. For example, other biocompatible materials may be used to manufacture the sheet of the prosthesis than those mentioned herein, as long as they are suitably bioinert for implantation within the eye, provide sufficient flexibility to permit folding of the sheet for insertion through an incision, and provide sufficient resilience to enable the sheet to substantially resume its shape prior to being folded.

Those of skill in the art will recognize that certain modifications of the embodiments disclosed herein can be made without exceeding the intended scope of the invention. Modifications to the geometry of the prosthesis 100, 200, the physical dimensions and the number of suture apertures can also be varied and will still be within the intended scope of the invention, as long as such geometries and dimensions provide sufficient points of contact that can produce the requisite stability of the prosthesis once implanted, as well as providing the requisite substantially centered alignment of the optical 55 or visual 59 axis of the eye with IOL optics 72, 82 captured thereon. For example, the geometry of the prosthesis could be hexagonal, pentagonal or even star shaped. Additional vertices could also be provided along the sides of rectangular prosthesis 100 without changing its geometry. The increased numbers of vertices of the geometry could provide additional suture apertures if desirable, which would lead to additional points of contact and greater stability. While the number of transscleral sutures 616 should be kept to a minimum to simplify the procedure, additional points of contact may be desirable.

The minimum points of contact necessary to prevent rotation of the prosthesis 100, 200 can be provided through at least two transscleral sutures 616 providing at least three points of contact between the sclera 36 of the eye 500 and prosthesis 100, 200 through apertures 102, 103 or 202, 203. Any lesser number could lead to undesired rotation of the implanted prosthesis, and therefore the IOL 70, 80, 870. When implanted as illustrated in FIG. 8, the three apertures 202, 203a, b of triangular embodiment 200 of the prosthesis 100, 200 (located proximally to its vertices) is an example of a geometry providing a minimum number of three points of contact. While there are actually four attachment points provided by the paired ends of the first and second looped transscleral sutures 616d, p, the points of contact as referred to herein are made with reference to the prosthesis 100, 200 itself. The second suture 616p provides two of the points of contact with the prosthesis 200, because it is looped through the two paired apertures 203a, b.

What is claimed is:

1. A capsular prosthesis for capturing and supporting an intraocular lens (IOL) within the posterior chamber of an eye, the intraocular lens having an optic and at least two haptics, said prosthesis further comprising:
a sheet of substantially bioinert material, the sheet having two substantially planar surfaces separated by the thickness of the material, the sheet further including:
a center aperture;
a plurality of suture apertures configured to receive at least two loops of suture, each of the loops having two ends,
wherein when the two ends of each of the at least two loops of suture are passed through the sclera of an eye approximately 180 degrees from one another along a predetermined surgical axis, the two loops of suture permitting adjustment of the sheet along the surgical axis to achieve centration of the center aperture to a predetermined axis of the eye, the centration becoming fixed when each of the two ends of the loops are then fixedly attached to the sclera.

2. The capsular prosthesis of claim 1 wherein the center aperture is configured to:
capture the optic of an IOL against a portion of a first one of the two planar surfaces, and
permit the haptics of the IOL to be passed through the center aperture to apply reactive force to a second one of the planar surfaces to retain the optic against the first surface and centered to the predetermined axis.

3. The capsular prosthesis of claim 2, wherein:
the center aperture defines at least two vertex features, each of the pair of vertex features being defined by the aperture and located proximally to one of the opposite ends of the sheet, and
when supporting an IOL thereon, each of the vertex features is configured to capture one of the haptics of the IOL as it passes through the center aperture to resist further movement of the haptic.

4. The capsular prosthesis of claim 1, wherein the sheet is substantially rectangular in geometry.

5. The capsular prosthesis of claim 4, wherein the at least three suture apertures include at least two pairs of suture apertures, one pair located at a proximal end of the sheet configured to receive the two ends of a proximal one of the at least two loops of suture, and a distal pair located at a distal end of the sheet configured to receive the two ends of a distal one of the two loops of suture.

6. The capsular prosthesis of claim 5, wherein each of the at least two pairs of suture apertures are located proximally to a different one of the four corners of the sheet.

7. The capsular prosthesis of claim 6, wherein each of the four corners of the sheet is rounded.

8. The capsular prosthesis of claim 1, wherein the sheet is substantially triangular in geometry.

9. The capsular prosthesis of claim 8, wherein the at least three suture apertures include at least one suture aperture located proximally to a different one of each vertex of the sheet, each suture aperture configured to receive at least one of the two ends of one of the at least two loops of suture.

10. The capsular prosthesis of claim 6, wherein each vertex of the sheet is rounded.

11. The capsular prosthesis of claim 1, wherein the bioinert material comprising the sheet is sufficiently deformable to permit folding of the sheet under a folding force during insertion into the eye through an incision, but sufficiently resilient such that it substantially unfolds back to its full geometry after the force is removed.

12. The capsular prosthesis of claim 1 wherein the bioinert material comprising the sheet is one of: silicone, polyimide, acrylic, polypropylene.

13. The capsular prosthesis of claim 2, wherein:
the IOL supported by the sheet is a three-piece IOL, and
the sheet is configured to support the three-piece IOL through reverse optic capture, wherein the optic is retained against a posterior one of the two planar surfaces of the sheet, and the haptics are passed through the center aperture to make contact with an anterior one of the two planar surfaces.

14. The capsular prosthesis of claim 1, wherein the sheet has a length of about 11 mm, a width of about 7 mm and a thickness of about 0.25 mm.

15. The capsular prosthesis of claim 15, wherein the center aperture has an inner length of about 8 mm between the vertex features and an internal width of about 5 mm.

16. The capsular prosthesis of claim 2, wherein the predetermined axis of the eye is the optical axis.

17. The capsular prosthesis of claim 2, wherein the predetermined axis of the eye is the visual axis.

18. The capsular prosthesis of claim 2, wherein:
the IOL supported by the sheet is a one-piece IOL, and
the sheet is configured to support the one-piece IOL through optic capture, wherein the optic is retained against an anterior one of the two planar surfaces of the sheet, and the haptics are passed through the center aperture to make contact with a posterior one of the two planar surfaces.

19. The capsular prosthesis of claim 18, wherein the IOL is a toric lens and the predetermined surgical axis is substantially equal to an axis of astigmatism of the eye.
